(19) United States
(12) Patent Application Publication
(10) Pub. No.: US 2014/0296949 A1
(43) Pub. Date: Oct. 2, 2014

(54) WARMING MOXIBUSTION DEVICE

(71) Applicant: Tsan-Ming CHEN, Dongshan (Yilan County) (TW)

(72) Inventor: Tsan-Ming CHEN, Dongshan (Yilan County) (TW)

(21) Appl. No.: 13/852,185

(22) Filed: Mar. 28, 2013

(51) Int. Cl. A61F 7/08

(52) U.S. Cl. CPC A61F 7/08; A61F 2007/006
USPC 607/107

(57) ABSTRACT

A warming moxibustion device is adapted for at least one moxa strip inserted therein for burning moxa to treat a patient. The warming moxibustion device includes a housing that has a first open end provided with an abutment surface to abut against a patient's body and a second open end opposite to the first open end. An insertion net is disposed in the housing and has two opposite sides respectively facing the first and second open ends. The insertion net has at least one insertion hole in size that is sufficient for insertion of the moxa strip therethrough.

### BACKGROUND OF THE INVENTION

1. Field of the Invention

The invention relates to a therapeutic device, and more particularly to a warming moxibustion device for burning moxa to treat a patient.

2. Description of the Related Art

Moxibustion is a kind of non-invasive combusting therapy in Chinese medicine that involves producing the moxa smoke by burning moxa. The moxa smoke is used to warm acupuncture points of a patient.

Referring to FIGS. 1 and 2, a conventional moxibustion device 1 as disclosed in Taiwanese Utility Model Patent No. M407068 includes a bell-shaped body 10, a cover 20, and a net 30. The bell-shaped body 10 has an upper opening 102, a lower opening 103 that is opposite to the upper opening 102, and a receiving space 101 that is in spatial communication with the upper opening 102 and the lower opening 103. The cover 20 removably covers the upper opening 102. The net 30 is disposed in the receiving space 101 proximate to the lower opening 103 so as to divide the receiving space 101 into a burning space 104 that allows the moxa to be burned therein and a moxibustion space 105 that corresponds to a region of the patient's body to be subjected to the moxibustion therapy. The net 30 also contributes to the prevention of moxa ashes from falling onto the patient. When the patient is in a prone or supine position during the moxibustion therapy, the burning moxa is placed into the burning space 104 through the upper opening 102. The cover 20 covers the upper opening 102. Then, the conventional moxibustion device 1 is disposed on a corresponding acupuncture point of the patient's body to carry out the moxibustion therapy.

Although the conventional moxibustion device 1 may be easily placed on the patient's body and may allow the burning moxa to maintain a predetermined distance from the patient for a long period of time during the moxibustion therapy, the conventional moxibustion device 1 implements the moxibustion therapy only to a single acupuncture point at a time. When the patient requires more than one acupuncture point to be subjected to the moxibustion therapy, a plurality of the conventional moxibustion devices 1 have to be simultaneously used, and the patient is frequently required to maintain the prone or supine position for a relatively longtime and to keep steady for preventing falling of the moxa ashes from the moxibustion devices 1. Hence, the patient tends to have muscle soreness and feel uncomfortable.

Therefore, there is still a need to further improve the treating time required for the conventional moxibustion device 1 to carry out the moxibustion therapy and the flexibility for the conventional moxibustion device 1 to allow the patient to change positions to a certain extent.

### SUMMARY OF THE INVENTION

Therefore, an object of the present invention is to provide a warming moxibustion device that can overcome the aforesaid drawbacks of the prior art.

According to the present invention, a warming moxibustion device adapted for at least one moxa strip inserted therein is used for burning moxa to treat a patient. The warming moxibustion device includes a housing and an insertion net.

The housing that has a first open end that is provided with an abutment surface to abut against a patient's body and a second open end that is opposite to the first open end.

The insertion net is disposed in the housing and has two opposite sides respectively facing the first and second open ends. The insertion net has at least one insertion hole for insertion of the moxa strip therethrough. The insertion hole has a size that is sufficient for insertion of the moxa strip.

### BRIEF DESCRIPTION OF THE DRAWINGS

Other features and advantages of the present invention will become apparent in the following detailed description of the preferred embodiments with reference to the accompanying drawings, of which:

FIG. 1 is a partly sectional perspective view for illustrating a conventional moxibustion device;

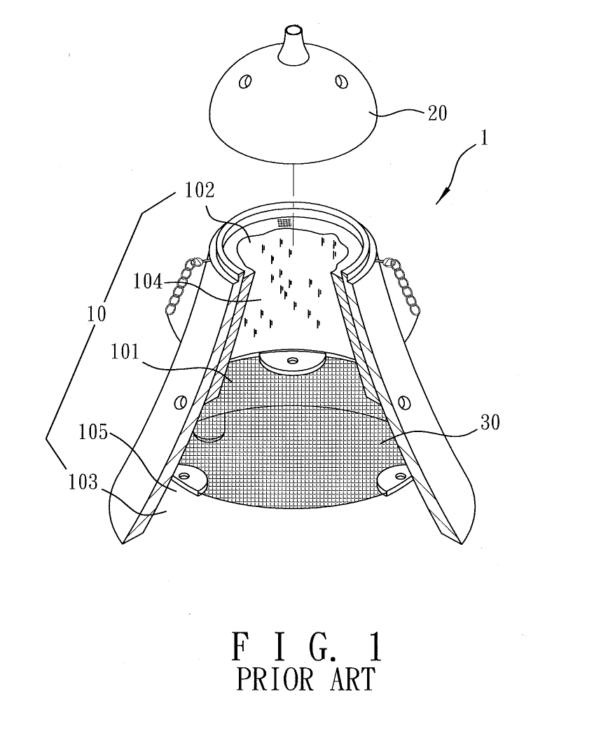

FIG. 2 is a schematic view for illustrating the conventional moxibustion device applied on a patient's back;

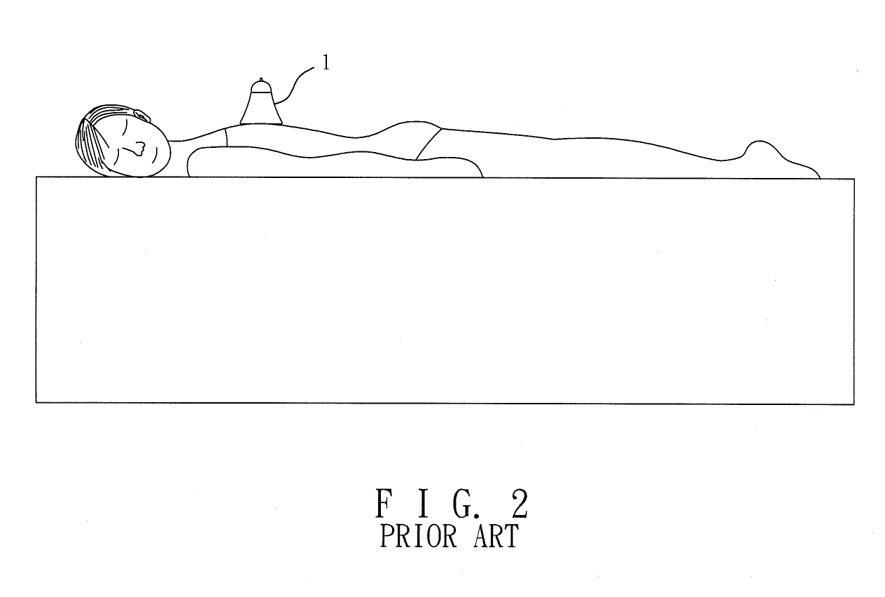

FIG. 3 is a perspective view for illustrating the first preferred embodiment of a warming moxibustion device according to the present invention;

FIG. 4 is a rear view of the first preferred embodiment of FIG. 3;

FIG. 5 is a partly enlarged rear perspective view of the first preferred embodiment of FIG. 3;

FIG. 6 is a rear perspective view for illustrating a varied insertion net of the first preferred embodiment;

FIG. 7 is a schematic view, illustrating the first preferred embodiment applied to a patient;

FIG. 8 is a rear view for illustrating the second preferred embodiment of a warming moxibustion device according to the present invention;

FIG. 9 is a partly enlarged view for illustrating an engagement between a cover and a guiding groove of FIG. 7; and

FIG. 10 is a perspective view for illustrating the third preferred embodiment of a warming moxibustion device according to the present invention.

### DETAILED DESCRIPTION OF THE PREFERRED EMBODIMENTS

Before the present invention is described in greater detail, it should be noted that like elements are denoted by the same reference numerals throughout the disclosure.

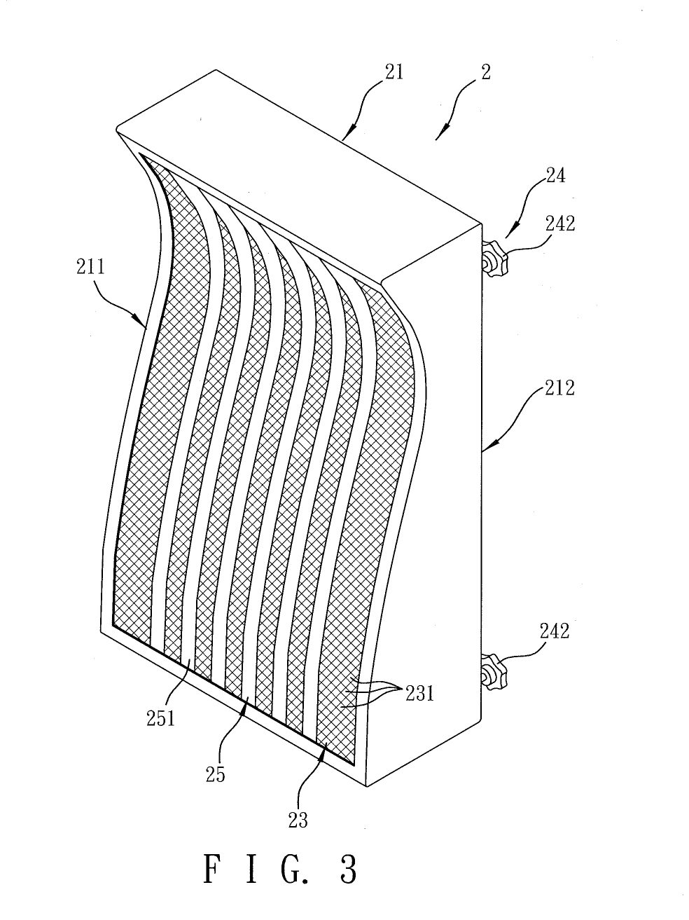

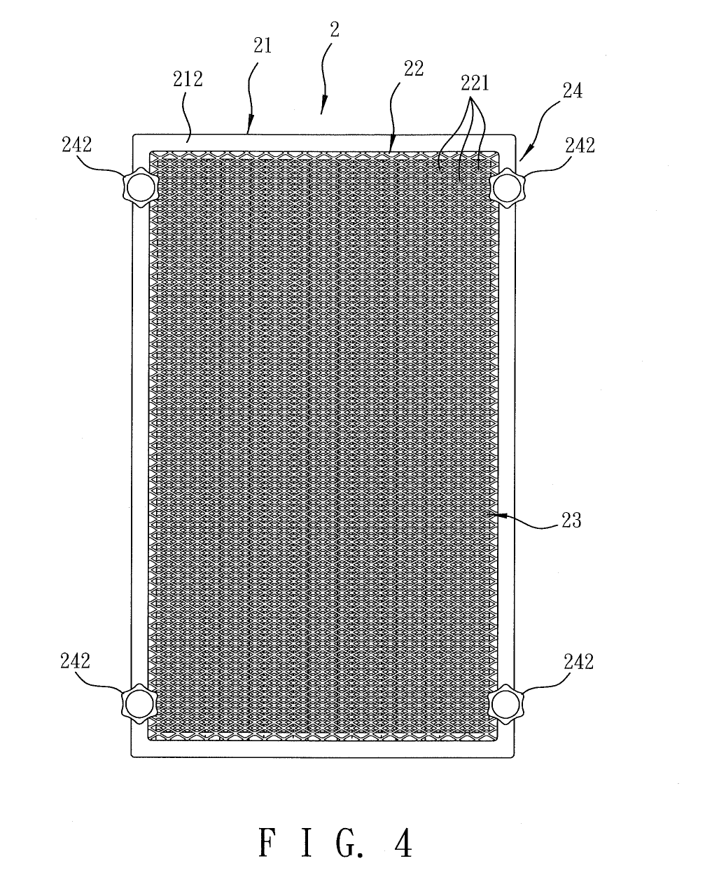

Referring to FIGS. 3 to 5 and 7, the first preferred embodiment of a warming moxibustion device 2 according to the present invention is adapted for at least one moxa strip 100 to be inserted therein for burning moxa to treat a patient. The warming moxibustion device 2 includes a housing 21 and an insertion net 22.

The housing 21 has a first open end 211 that is provided with an abutment surface 251 to abut against the patient' s body and a second open end 212 that is opposite to the first open end 211. The abutment surface 251 is a curved surface adapted for conforming to a portion of the patient's body that abuts against the abutment surface 251. In this embodiment, the abutment surface 251 has a shape conformed to the patient's back. However, the present invention is not limited in this respect.

The insertion net 22 is disposed in the housing 21 between the first and second open ends 211, 212 and has two opposite sides respectively facing the first and second open ends 211, 212. The insertion net 22 has at least one insertion hole 221 that has a size large sufficient for insertion of the moxa strip 100 therethrough. In this preferred embodiment, the insertion net 22 is proximate to and substantially parallel to a cross-section of the second open end 212.

Preferably, the warming moxibustion device 2 further includes an isolation net 23 that is mounted to the housing 21 in proximity to the abutment surface 251. In this embodiment, the insertion net 22 is disposed between the isolation net 23 and the second open end 212. The isolation net 23 has a plurality of isolation holes 231. The insertion net 22 has a plurality of insertion holes 221 for a plurality of the moxa strips 100 to be inserted therethrough, respectively. The isolation holes 231 are staggered with respect to the insertion holes 221. Preferably, each of the isolation holes 231 of the isolation net 23 has a size smaller than that of a cross-section of the moxa strip 100, thereby impeding the moxa strip 100 to pass therethrough to hurt the patient. Hence, safety of the patient during warming moxibustion therapy can be ensured. Alternatively, the isolation holes 231 may have a size similar to that of the insertion holes 221 so that the manufacturer may unify the specification of the isolation holes 231 of the isolation nets 23 with the insertion holes 221 of the insertion net 22 to reduce fabrication cost. However, it should be noted that the isolation holes 231 have to be staggered with respect to the insertion holes 221 when the isolation net 23 and the insertion net 22 are disposed, so that the patient is protected from getting burned by the burning moxa strip 100 during the warming moxibustion therapy.

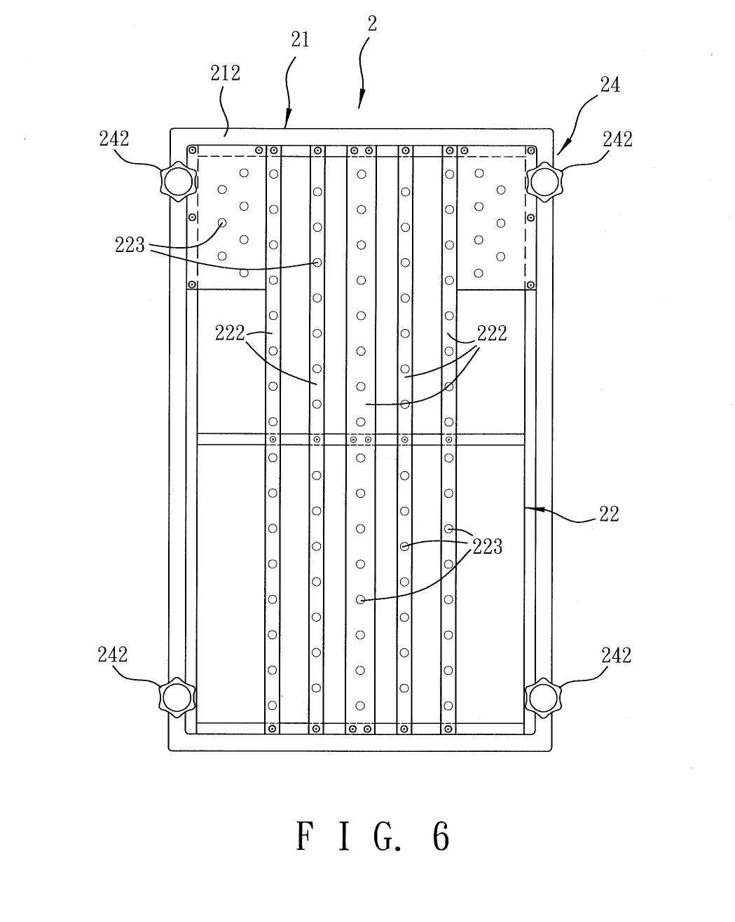

In the first preferred embodiment, the insertion net 22 is configured as a one-piece wire screen. However, the insertion net 22 of this invention is not limited to the configuration exemplified in the first preferred embodiment and may be configured to other shapes, as long as the moxa strips 100 are able to be inserted into and fixed on the insertion net 22 for conducting the warming moxibustion therapy on the acupuncture points of the patient. For example, as shown in FIG. 6, the insertion net 22 may be composed of a plurality of separately perforated steel slats 222 that respectively have a plurality of through holes 223. Each of the through holes 223 is adapted to allow insertion of the moxa strip 100 therethrough.

Preferably, the warming moxibustion device 2 further includes an adjusting unit 24 that is connected to the insertion net 22 and that is operable to vary a distance between the insertion net 22 and the isolation net 23.

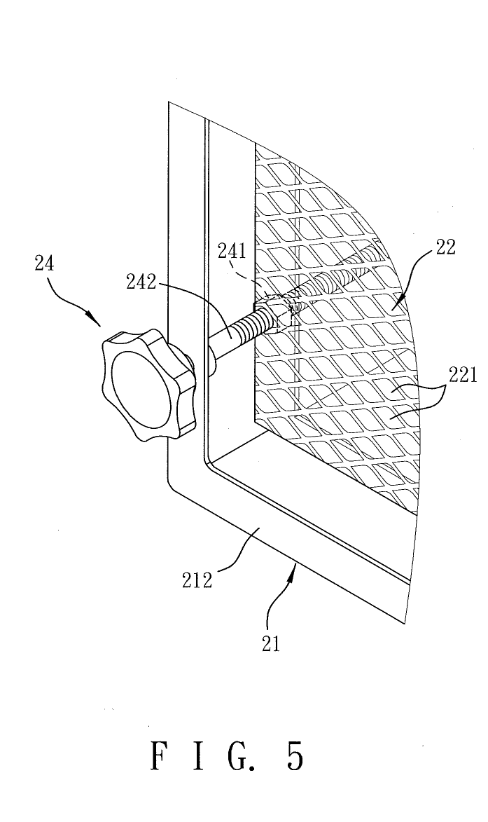

Referring to FIG. 5, the adjusting unit 24 includes a plurality of screw rods 242 rotatably and non-threadedly connected to the housing 21, and a plurality of fixtures 241 each disposed threadedly around a corresponding one of the screw rods 242 and connected to the insertion net 22. The insertion net 22 is moved toward or away from the isolation net 23 when the screw rods 242 are rotated. In this first preferred embodiment, each of the screw rods 242 is a headed screw rod. Each of the fixtures 241 is configured as a nut. The movement of the insertion net 22 relative to the isolation net 23 is linked to the rotation of the screw rods 242 with respect to the fixtures 241 so as to adjust the distance between the insertion net 22 and the isolation net 23. By virtue of an adjustable distance between the insertion net 22 and the isolation net 23, the distance between the burning moxa strips 100 inserted into the insertion net 22 and the patient is also adjustable so as to conduct the warming moxibustion at a relatively effective and safe distance.

Preferably, the warming moxibustion device 2 further includes a backrest 25 that is disposed at the first open end 211 of the housing 21. The backrest 25 has the abutment surface 251. In this preferred embodiment, the backrest 25 is composed of a plurality of spaced apart curved slats, each shaped to a predetermined configuration. The abutment surface 251 conforms in shape to the back of the patient's body.

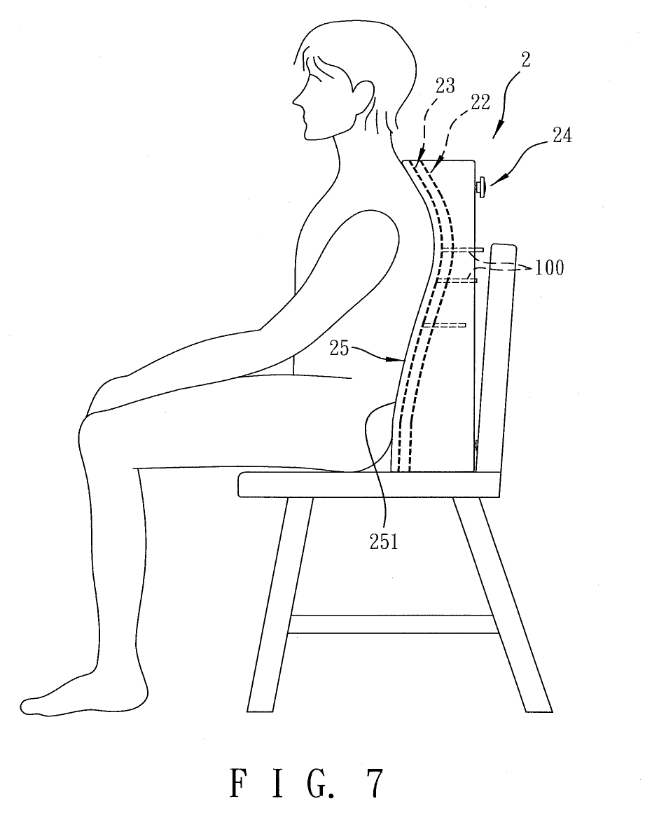

With further reference to FIG. 7, when the warming moxibustion device 2 is used for performing moxibustion therapy, the patient may at first comfortably lean on the abutting surface 251 of the backrest 25. Then, a therapist inserts the burning moxa strips 100 into the respective insertion holes 221 of the insertion net 22 that corresponds to the acupuncture points of the patient's body for starting moxibustion. The isolation net 23 prevents the burning moxa strips 100 from being in direct contact with the patient, so that the moxibustion therapy can be safely performed and accidental burns can be avoided. When the burning moxa strips 100 become shortened, the therapist may simply rotate the screw rods 242 of the adjusting unit 24 to adjust the distance between the insertion net 22 and the isolation net 23 to maintain the distance between the burning moxa strips 100 and the patient. Therefore, the efficiency of the moxibustion therapy can be optimized.

In alternative embodiments, the size of the isolation holes 231 of the isolation net 23 may be reduced so that ashes of the burning moxa strip 100 are hindered to pass through the isolation holes 231. The warming moxibustion device 2 allows the patient to safely rest in one of the prone and supine positions. Therefore, the warming moxibustion device 2 may simultaneously perform the moxibustion therapy on and stimulate a plurality of acupuncture points respectively located in the patient's back, chest, or abdomen portion. It is noted that the warming moxibustion device 2 can be applied in other ways than those previously exemplified.

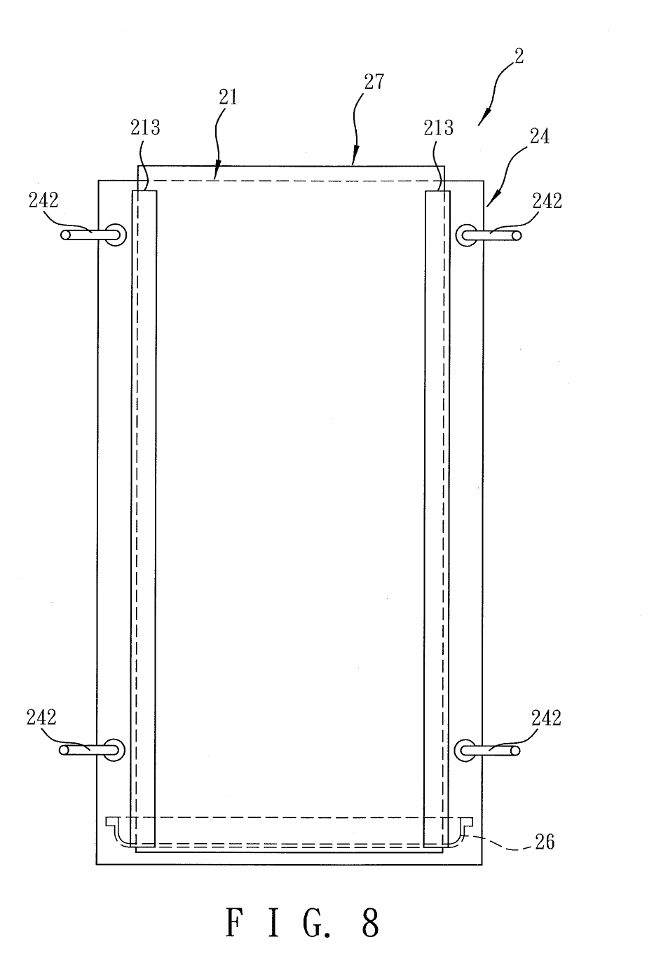

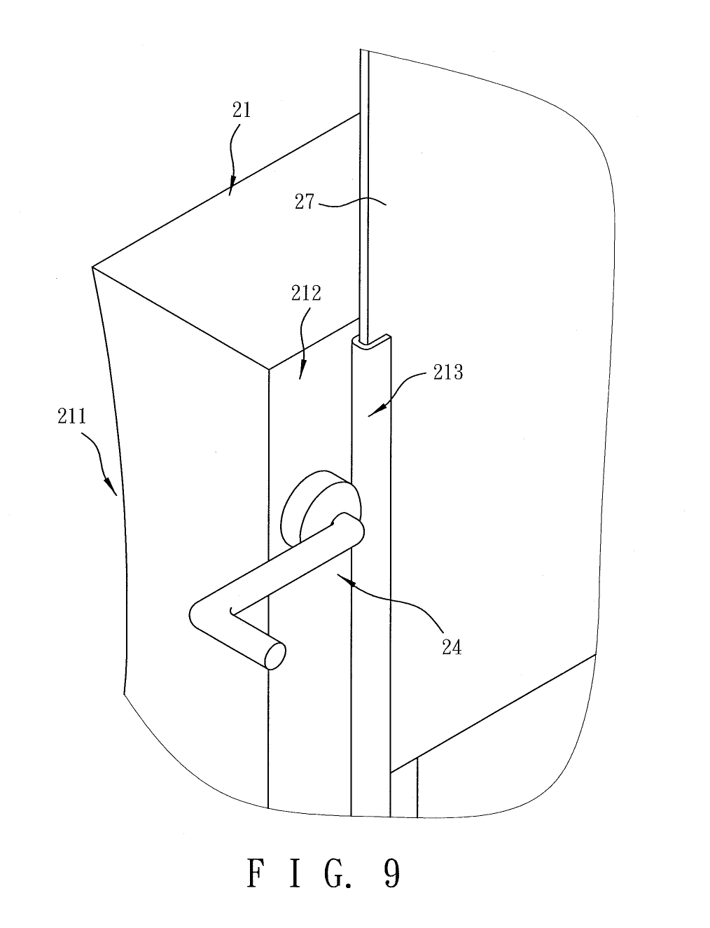

Referring to FIGS. 8 and 9, the second preferred embodiment of a warming moxibustion device 2 according to the present invention has a structure similar to that of the first preferred embodiment except for the warming moxibustion device 2 further including an ash tray 26 and a cover plate 27.

The ash tray 26 is detachably mounted on the housing 21 for collecting ashes from the burning moxa strip 100 and is configured as a substantially rectangular tray. The ash tray 26 may be configured to have some other shapes, such as a slidable drawer and an engaging plate. Since the feature of this invention does not reside in the configuration of the ash tray 26, which may readily be appreciated by those skilled in the art, details of the same are omitted herein for the sake of the brevity. The cover plate 27 is mounted on the housing 21 and covers the second open end 212.

In this embodiment, the housing 21 further includes two opposite spaced-apart guiding grooves 213 that are disposed at the second open end 212 of the housing 21 and that are configured to comply with the dimension of the cover plate 27 so that the cover plate 27 is slidably inserted into the guiding grooves 213. When the burning moxa strips 100 produce smoke during the moxibustion therapy, the cover plate 27 may close the second open end 212 to prevent the smoke from dispersing out of the housing 21. Therefore, the smoke may be retained in the housing 21 without causing annoyance to other people sharing the moxibustion therapy environment.

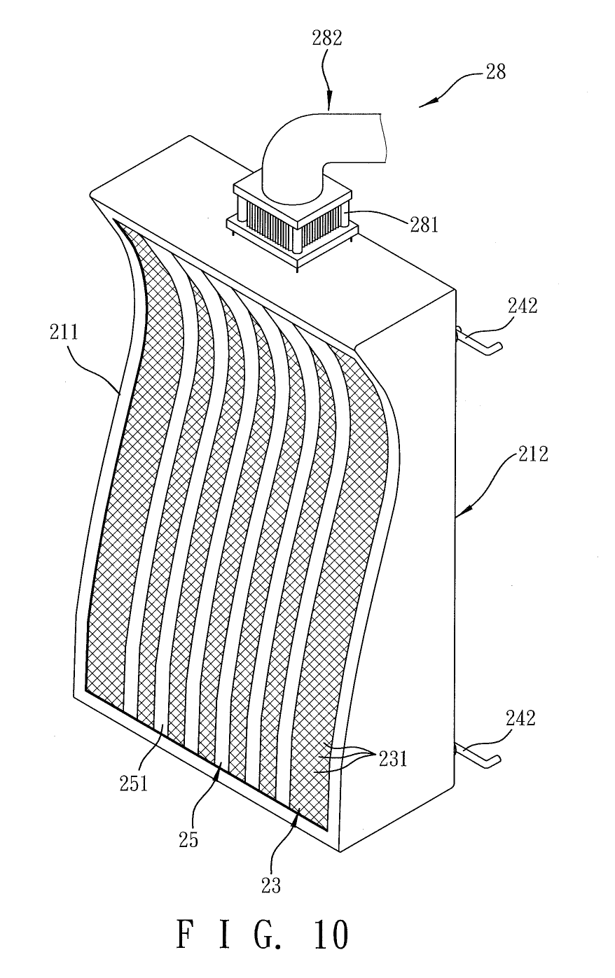

FIG. 10 shows the third preferred embodiment of a warming moxibustion device 2 according to the present invention, which has a structure generally similar to that of the second preferred embodiment except for the warming moxibustion device 2 further including an exhaust unit 28 that is mounted on the housing 21. The exhaust unit 28 includes an exhaust fan 281 that is disposed on a top side of the housing 21 and that communicates with an interior of the housing 21, and an exhaust pipe 282 that is connected to the exhaust fan 281. When the exhaust fan 281 is operated, the smoke of the burning moxa strips 100 is outwardly expelled from the housing 21 through the exhaust pipe 282.

By virtue of an installation of the exhaust unit 28 on the housing 21 of the warming moxibustion device 2, the smoke produced from the burning moxa strips 100 may be gathered and expelled outwardly by the exhaust fan 281 through the exhaust pipe 282, which prevents the smoke from annoying other people sharing the moxibustion therapy environment.

To sum up, by virtue of coordination among the housing 21, the insertion net 22, and the moxa strips 100, the warming moxibustion device 2 of this invention allows the patient to change physical positions to avoid any physical discomfort from maintaining the same position during long moxibustion therapy time and is capable of being implemented simultaneously on different acupuncture points of the patient's body. In addition, since the abutment surface 251 of the housing 21 is configured to conform to the patient's body, the patient will have an enhanced comfort feeling. Furthermore, the ash tray 26 is able to collect ashes of the burning moxa strips 100, facilitating cleaning of the warming moxibustion device 2 after completion of moxibustion therapy. Moreover, on the housing 21, the exhaust unit 28 may outwardly expel smoke produced from the burning moxa strips 100 to avoid annoyance to other people sharing the moxibustion therapy environment.

While the present invention has been described in connection with what are considered the most practical and preferred embodiments, it is understood that this invention is not limited to the disclosed embodiments but is intended to cover various arrangements included within the spirit and scope of the broadest interpretation so as to encompass all such modifications and equivalent arrangements.

What is claimed is:

1. A warming moxibustion device adapted for at least one moxa strip inserted therein for burning moxa to treat a patient, said warming moxibustion device comprising:
a housing that has a first open end provided with an abutment surface to abut against a patient's body and a second open end opposite to said first open end; and
an insertion net that is disposed in said housing and that has two opposite sides respectively facing said first and second open ends, said insertion net having at least one insertion hole for insertion of the moxa strip therethrough, said insertion hole having a size that is sufficient for insertion of the moxa strip.

2. The warming moxibustion device as claimed in claim 1, wherein said abutment surface is a curved surface substantially conforming to a portion of the patient's body.

3. The warming moxibustion device as claimed in claim 1, further comprising an isolation net that is mounted to said housing in proximity to said abutment surface, said insertion net being disposed between said isolation net and said second open end.

4. The warming moxibustion device as claimed in claim 3, wherein said isolation net has a plurality of isolation holes, said insertion net having a plurality of said insertion holes, said isolation holes are staggered with respect to said insertion holes.

5. The warming moxibustion device as claimed in claim 3, wherein said isolation net has a plurality of isolation holes, each of said isolation holes of said isolation net having a size smaller than a cross-section of the moxa strip for impeding the moxa strip to pass therethrough.

6. The warming moxibustion device as claimed in claim 3, further comprising an adjusting unit connected to said insertion net and operable to vary a distance between said insertion net and said isolation net.

7. The warming moxibustion device as claimed in claim 6, wherein said adjusting unit includes a plurality of screw rods rotatably and non-threadedly connected to said housing, and a plurality of fixtures each disposed threadedly around a corresponding one of said screw rods and connected to said insertion net, said insertion net being movable toward or away from said isolation net when said screw rods are rotated.

8. The warming moxibustion device as claimed in claim 6, further comprising a backrest disposed at said first open end, said backrest having said abutment surface.

9. The warming moxibustion device as claimed in claim 8, wherein said abutment surface conforms in shape to a back of the patient's body.

10. The warming moxibustion device as claimed in claim 1, further comprising an ash tray that is detachably mounted on said housing for collecting ashes from the burning moxa strip.

11. The warming moxibustion device as claimed in claim 10, further comprising a cover plate that is mounted on said housing, and that covers said second open end.

12. The warming moxibustion device as claimed in claim 11, wherein said housing further includes two opposite spaced-apart guiding grooves disposed at said second open end of said housing, said cover plate being slidably inserted into said guiding grooves.

13. The warming moxibustion device as claimed in claim 11, further comprising an exhaust unit mounted on said housing, said exhaust unit including an exhaust fan that is disposed on a top side of said housing, and an exhaust pipe that is connected to said exhaust fan.
